(19) United States
(12) Patent Application Publication
(10) Pub. No.: US 2011/0201060 A1
(43) Pub. Date: Aug. 18, 2011

(54) PROCESS FOR THE PREPARATION OF SCYLLO-INOSITOL

(75) Inventors: Rajarathnam E. Reddy, Gurnee, IL (US); Sanjay R. Chemburkar, Gurnee, IL (US); Douglas R. Spaulding, Green Oaks, IL (US); Yi Pan, Gurnee, IL (US); Lei Cao, Lake Bluff, IL (US); Jose A. Restituyo, Gurnee, IL (US); Richard Lorenzini, Antioch, IL (US); Michael DeMarco, Wadsworth, IL (US)

(73) Assignee: Abbott Laboratories, Abbott Park, IL (US)

(21) Appl. No.: 13/026,749

(22) Filed: Feb. 14, 2011

Related U.S. Application Data
(60) Provisional application No. 61/304,581, filed on Feb. 15, 2010.

(51) Int. Cl. C12P 19/02

(52) U.S. Cl. 435/105

(57) ABSTRACT

This invention pertains to a process for manufacturing scyllo-Inositol. Specifically, the current invention pertains to a process for converting myo-Inositol to scyllo-Inositol using a bioconversion process.

### CROSS-REFERENCE TO RELATED APPLICATIONS

This application claims priority to U.S. provisional application Ser. No. 61/304,581, filed Feb. 15, 2010, the contents of which are hereby incorporated by reference in its entirety.

### FIELD OF THE INVENTION

This invention pertains to a process for manufacturing scyllo-Inositol. Specifically, the current invention pertains to a process for converting myo-Inositol to scyllo-Inositol using a bioconversion process.

### BACKGROUND OF THE INVENTION

Scyllo-Inositol (1) [CAS Registry No. 488-59-5] is one of the nine stereoisomers of hexahydrocyclohexane, found to be present in a variety of natural sources. However, it is present in only small quantities (Martinez-Castro et al. Food Chem. 2004, 87, 325) when compared to myo-Inositol (2) [CAS Registry No. 87-89-8], a widely used nutritional supplement. Scyllo-Inositol (1) also is present in a variety of mammalian tissues (Sherman, et al. Biochemistry 1968, 7, 819) and at elevated concentrations in the brain of individuals suffering from Alzheimer's disease (Michaelis et al. NMR Biomed. 1993, 6, 105; Griffith et al. Ibid 2007, 20, 709). Further, it has been demonstrated that scyllo-Inositol (1) is able to interact with most neurotoxic components (e.g., Aβ42 peptide) of senile plaques that are deposited in individuals suffering with Alzheimer's disease and induces change in its secondary structure, stabilizes small Aβ-oligomers, and completely blocks the fibril formation (McLaurin et al. J. Mol. Biol. 1998, 278, 183; McLaurin et al. Ibid 2000, 275, 18495; Fenili et al. Ibid 2007, 85, 603, and WO

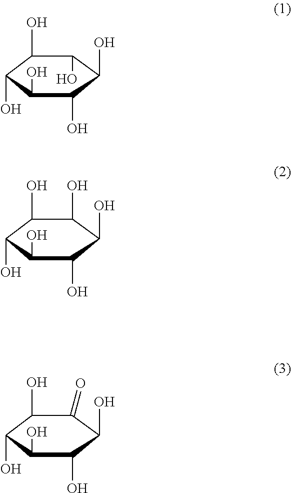

2004/075882). As a result of these research findings, the scyllo-Inositol (1) is currently undergoing further clinical studies to evaluate its efficacy and determine its usefulness for the treatment of Alzheimer's disease (Wolfson, Chem. Biol. 2008, 89).

A variety of methods for the preparation of scyllo-Inositol (1) have been reported. One method of preparation is based on an enzymatic approach on 2,4,6/3,5-pentahydroxy cyclohexanone (meso-2-inosose), which was prepared from myo-Inositol (2) using Acetobacter suboxydans (Kluyver et al. Rec. Trav. Chim. Pays-Bas. 1939, 58, 956), while another method of preparation is via reduction of meso-2-inosose by sodium amalgam reagent in an acidic medium followed by separation (Posternak, Helv. Chim. Acta 1941, 24, 1045) or using a sodium borohydride reducing agent (Reymond, Helv. Chim. Acta 1957, 40 492) in poor yield. Another method of preparation is based on starting from hexahydroxybenzene in low yield (Angyal et al., J. Chem. Soc. 1957, 3682) or utilizing conduritol as a raw material (Nakajima et al., Chem. Ber. 1959, 92, 173). An additional method of preparation is by separation on an ion exchange resin (Sasaki, et al. Carbohydrate Res. 1987, 167, 171) or chromatographic separation through complexation (Sasaki, et al. Carbohydrate Res. 1988, 183, 1) or through chemical resolution on a silica HPLC column (Ghias-ud-din et al., J. Chromatogr. 1981, 211, 295). A further method starts with myo-Inositol (2) via oxidation followed by reduction of the resulting myo-inososepetaacetate and hydrolysis (Kohne et al. Liebigs Ann. Chem. 1985, 4, 866; DE 1985/3405663), while another method starts with myo-Inositol (2) via equilibration using Raney nickel under basic conditions (Husson et al., Carbohydrate Res. 1998, 307, 163), while other methods incorporate palladium catalysts for sequential cycloaddition reactions, starting from 6-O-acetyl-5-enopyranosides (Takahashi et al., J. Org. Chem. 2001, 66, 2705). Additional methods of preparation have been demonstrated through the use of myo-Inositol (2) via selective protection using sulfonate groups followed by oxidation and reduction (Sarmah et al., Carbohydrate Res. 2003, 338, 998). Alternatively, via scyllo-inosose (3), a method of preparation involves starting with myo-Inositol (2) using Pseudomos and Acetobacter (Kenji et al., JP 2003/102492). Other methods of preparation include starting with conduritol, which in turn was prepared from benzoquinone (Bolck et al., Eur. J. Org. Chem. 2003. 10, 1958): via scyllo-inosose (3), which was prepared from myo-Inositol (2), based on use of organo-silyl groups for protection followed by chemical or enzymatic reduction (Kenji et al., JP 2003/107287 or via enzymatic reduction of scyllo-inosose (3) (Kenji et al., WO 2005/035774); or from myo-Inositol (2) (Cruz et al., WO 2007/119108).

Among these, only limited methods are suitable for preparation of scyllo-Inositol (1) on a larger scale, particularly in high quality suitable for pharmaceutical applications. However, the method described by Kenji et al. (WO 2005/035774), which is based on the conversion of myo-Inositol (2) to scyllo-inosose (3) using microorganisms belonging to the genus Acetobacter followed by enzymatic reduction of scyllo-inosose (3) to scyllo-Inositol (1), has been shown to be amenable for kilogram scale operations.

In the method described in the above paragraph, myo-Inositol (2) is first converted to scyllo-inosose (3), which is then transformed to scyllo-Inositol (1) in major percentage, but leaves a significant portion of scyllo-inosose (3) unreacted, resulting in a mixture. Further, a significant amount of scyllo-quercitol (6) is also formed as a by-product in this transformation. In order to separate the desired scyllo-Inositol (1) from the mixture of unreacted scyllo-inosose (3) and by-product scyllo-quercitol (6), Kenji et al. (WO 2005/035774) has used first cell separation followed by chemical transformation of scyllo-Inositol (1) to scyllo-Inositol-diborate-disodium complex (SBC salt, 5) followed by hydrolysis using hydrochloric acid in a mixture of methanol and water. This transformation requires use of boric acid to form SBC salt (5) (Weissbach, J. Am. Chem. Soc. 1957, 23, 329 and Grainger, Acta Cryst. 1981, B37 563) and use of concentrated hydrochloric acid, which is not only corrosive to the equipment, but requires special operating procedures to ensure safety from hazardous fumes. In addition, formation or presence of any residual amount of both D and L-chiro-Inositols (7 and 8), which are related substance impurities in scyllo-Inositol (1) drug substance, has not been determined.

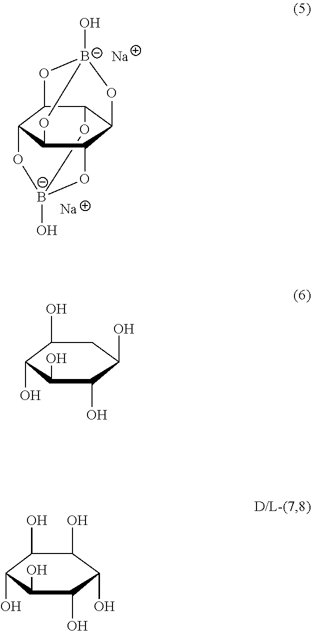

Further, since the method requires use of boric acid during the recovery of scyllo-Inositol (1) for chemoselective derivatization to SBC salt (5), it is critical that the boric acid is removed in subsequent steps to the appropriate levels in final drug substance per ICH and regulatory guidelines. In addition, the fact that unreacted scyllo-inosose (3), which is present in significant quantities at the end of biotransformation step, is destroyed through base and thermal degradation, the potential yield loss from this operation is inevitable. Therefore, with respect to reagents, overall yield, and ensuring the product quality, there is a need for improved methods of manufacturing scyllo-Inositol (1).

### SUMMARY OF THE INVENTION

The present invention provides an efficient and safe approach for the preparation of scyllo-Inositol (1). In one embodiment of the current invention, scyllo-Inositol is produced according to the following process:

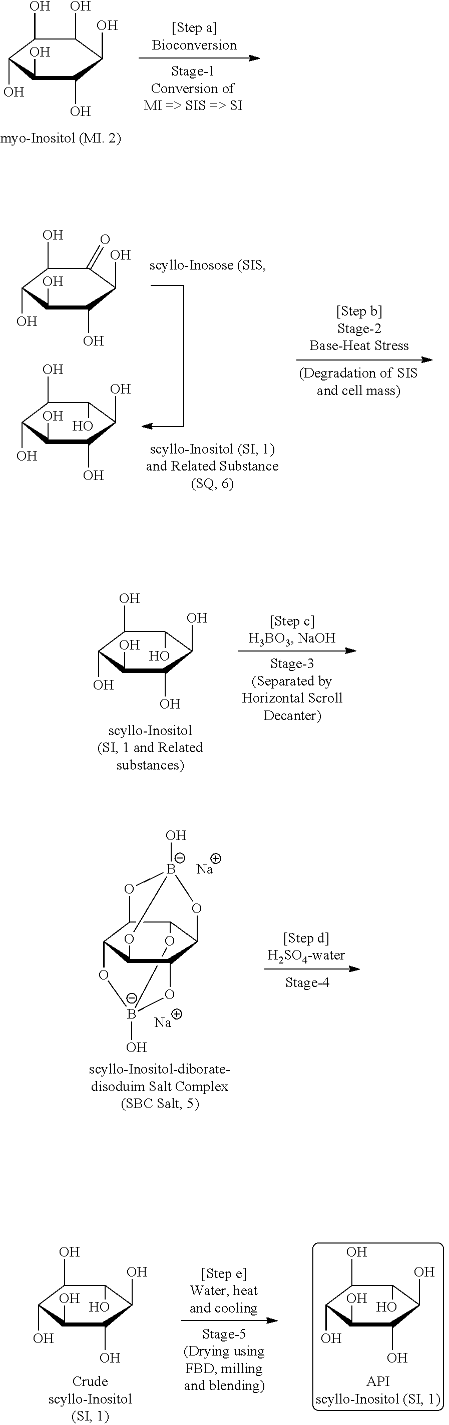

In another embodiment, the current invention comprises a process for preparing scyllo-Inositol (1) comprising the steps of: (a) subjecting myo-Inositol (2) to a bioconversion process to produce scyllo-Inosose (3) and scyllo-Inositol (1); (b) reacting the scyllo-Inosose and scyllo-Inositol produced in step (a) with a basic compound and heating to degrade the scyllo-Inosose and lyse the cell mass; (c) converting the scyllo-Inositol of step (b) to scyllo-Inositol-diborate-disodium salt complex using boric acid and sodium hydroxide; (d) hydrolyzing the scyllo-Inositol-diborate-disodium salt complex to produce crude scyllo-Inositol; and (e) crystallizing the crude scyllo-Inositol to produce crystalline scyllo-Inositol. The bioconversion of step (a) may comprise a fermentation, whereby the fermentation is facilitated by a microorganism capable of converting the myo-Inositol into scyllo-Inositol. Microorganisms capable of converting myo-Inositol into scyllo-Inositol comprises Acetobacter cerevisiae, Acetobacter malorum, Acetobacter orleanensis, Acetobacter indonesiensis, Acetobacter orientalis, Acetobacter aceti, Acetobacter liquefaciens, Acetobacter pasteurianus, Acetobacter hansenii, Burkholderia andropogonis, Burkholderia caryophylli, and Burkholderia graminis. Generally, the microorganism capable of converting the myo-Inositol into scyllo-Inositol is provided in the form of a lyophilized or frozen culture. Step (a) of the current process is performed at a temperature ranging from about 20° C. to about 40° C. In another embodiment, step (a) is performed at a temperature ranging from about 26° C. to about 30° C.

The basic compound of step (b) may comprise sodium hydroxide, potassium hydroxide, sodium carbonate, calcium carbonate, and combinations thereof. The amount of basic compound added to the fermentation mixture in step (b) is generally an amount sufficient to increase the pH of the fermentation mixture to a level ranging from about 10 to about 13. In another embodiment, the amount of basic compound added to the fermentation mixture in step (b) is an amount sufficient to increase the pH of the fermentation mixture to a level ranging from about 12 to about 13. The heating process of step (b) typically comprises a direct steam injection to increase the temperature of the fermentation mixture, increasing the temperature to a level ranging from about 100° C. to about 150° C. In another embodiment, the temperature of the fermentation mixture is increased to a level ranging from about 115° C. to about 130° C. In additional embodiments, the reaction mixture produced by step (b) may subsequently be cooled to a temperature less than about 80° C.

The reaction of step (c) is typically performed at a temperature ranging from about 60° C. to about 80° C. The amount of sodium hydroxide incorporated into the reaction mixture of step (c) is generally sufficient to establish a pH ranging from about 8.5 to about 11. In another embodiment, the amount of sodium hydroxide incorporated into the reaction mixture of step (c) is sufficient to establish a pH ranging from about 9.5 to about 10.5. Step (c) may further comprise the subsequent cooling of the reaction mixture to a temperature of less than 30° C. The scyllo-Inositol-diborate-disodium salt complex produced by step (c) may subsequently be passed through a horizontal scroll decanter prior to step (d).

In step (d), the combination of scyllo-Inositol-diborate-disodium salt complex and water is heated to a temperature ranging from about 30° C. to about 50° C., prior to addition of sulfuric acid. In another embodiment, the combination of scyllo-Inositol-diborate-disodium salt complex and water in step (d) is heated to a temperature ranging from about 36° C. to about 43° C., prior to addition of sulfuric acid. The amount of sulfuric acid added to the combination of scyllo-Inositol-diborate-disodium salt complex and water in step (d) is sufficient to decrease the pH to a level ranging from about 2 to about 3.5. The reaction product of step (d) may subsequently be cooled to a temperature ranging from about 15° C. to about 26° C. In another embodiment, the reaction product of step (d) may subsequently be cooled to a temperature ranging from about 18° C. to about 24° C.

Step (e) typically comprises the addition of water to the crude scyllo-Inositol produced by step (d), followed by heating of the reaction mixture, and subsequent cooling to produce the crystalline scyllo-Inositol. Specifically, subsequent to the addition of water to the crude scyllo-Inositol produced by step (d), the reaction mixture of water and crude scyllo-Inositol is heated to a temperature ranging from about 70° C. to about 100° C. In another embodiment, the reaction mixture of water and scyllo-Inositol may be heated to a temperature ranging from about 85° C. to about 95° C. The reaction mixture of water and crude scyllo-Inositol produced in step (e) is subsequently cooled to a temperature ranging from about 8° C. to about 16° C. Generally, after cooling, the solution of crude scyllo-Inositol and water produced in step (e) is subjected to a solid separation process by either solid filtration or centrifugation, and drying to produce crystalline scyllo-Inositol. In one embodiment, the solid separation process may comprise basket centrifugation and a scrolled decanter centrifuge. Moreover, in another embodiment, the drying process may comprise the use of hot air in a fluid bed dryer, a tray dryer, a tumble dryer, and a unidryer.

In a further embodiment, the current invention comprises a process for producing myo-Inositol, without the production of the scyllo-Inositol-diborate-disodium salt complex intermediate, according to the following steps:

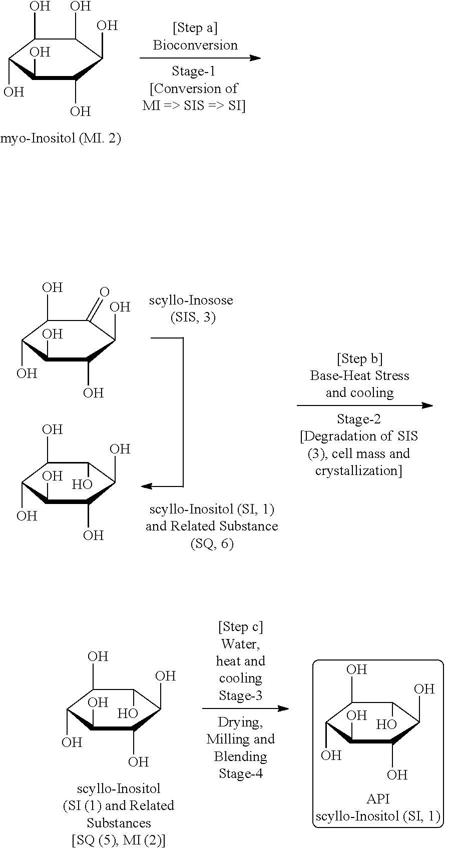

Specifically, the current invention comprises a process for preparing scyllo-Inositol (1) comprising the steps of: (a) subjecting myo-Inositol (2) to a bioconversion process to produce scyllo-Inosose (3) and scyllo-Inositol (1); (b) reacting the scyllo-Inosose and scyllo-Inositol produced in step (a) with a basic compound and heating to degrade the scyllo-Inosose and lyse the cell mass; and (c) crystallizing the crude scyllo-Inositol to produce crystalline scyllo-Inositol. The bioconversion of step (a) may comprise a fermentation, whereby the fermentation is facilitated by a microorganism capable of converting the myo-Inositol into scyllo-Inositol. Microorganisms capable of converting myo-Inositol into scyllo-Inositol comprises Acetobacter cerevisiae, Acetobacter malorum, Acetobacter orleanensis, Acetobacter indonesiensis, Acetobacter orientalis, Acetobacter aceti, Acetobacter liquefaciens, Acetobacter pasteurianus, Acetobacter hansenii, Burkholderia andropogonis, Burkholderia caryophylli, and Burkholderia graminis. Generally, the microorganism capable of converting the myo-Inositol into scyllo-Inositol is provided in the form of a lyophilized, frozen culture. Step (a) of the current process is performed at a temperature ranging from about 20° C. to about 40° C. In another embodiment, step (a) is performed at a temperature ranging from about 26° C. to about 30° C.

The basic compound of step (b) may comprise sodium hydroxide, potassium hydroxide, sodium carbonate, calcium carbonate, and combinations thereof. The amount of basic compound added to the fermentation mixture in step (b) is generally an amount sufficient to increase the pH of the fermentation mixture to a level ranging from about 10 to about 13. In another embodiment, the amount of basic compound added to the fermentation mixture in step (b) is an amount sufficient to increase the pH of the fermentation mixture to a level ranging from about 12 to about 13. The heating process of step (b) typically comprises a direct steam injection to increase the temperature of the fermentation mixture, increasing the temperature to a level ranging from about 100° C. to about 150° C. In another embodiment, the temperature of the fermentation mixture is increased to a level ranging from about 115° C. to about 130° C. In additional embodiments, the reaction mixture produced by step (b) may subsequently be cooled to a temperature less than about 80° C.

Step (c) typically comprises the addition of water to the crude scyllo-Inositol produced by step (b), followed by heating of the reaction mixture, and subsequent cooling to produce the crystalline scyllo-Inositol. The reaction mixture of water and crude scyllo-Inositol produced in step (c) is subsequently cooled to a temperature ranging from about 8° C. to about 16° C. Generally, after cooling, the solution of crude scyllo-Inositol and water produced in step (c) is subjected to a solid separation process by either solid filtration or centrifugation, and drying to produce crystalline scyllo-Inositol. In one embodiment, the solid separation process may comprise basket centrifugation and scrolled decanter centrifugation. Moreover, in another embodiment, the drying process may comprise the use of hot air in a fluid bed dryer, a tray dryer, a tumble dryer, and a unidryer.

### DESCRIPTION OF THE DRAWINGS

Other advantages of the present invention will be readily appreciated, as the same becomes better understood by reference to the following detailed description when considered in connection with the accompanying drawings, wherein:

FIG. 1 illustrates the commercial scale process of the current invention. Specifically, FIG. 1 illustrates the process for converting myo-Inositol to scyllo-Inositol by the subsequent steps of bioconversion; degradation by base and heat stress applied to the fermentation mixture; reaction with boric acid and sodium hydroxide to produce scyllo-Inositol-diborate-disodium salt complex; hydrolysis of the scyllo-Inositol-diborate-disodium salt complex by reaction with sulfuric acid and water to produce crude scyllo-Inositol; and the crystallization of the crude scyllo-Inositol to produce crystalline scyllo-Inositol.

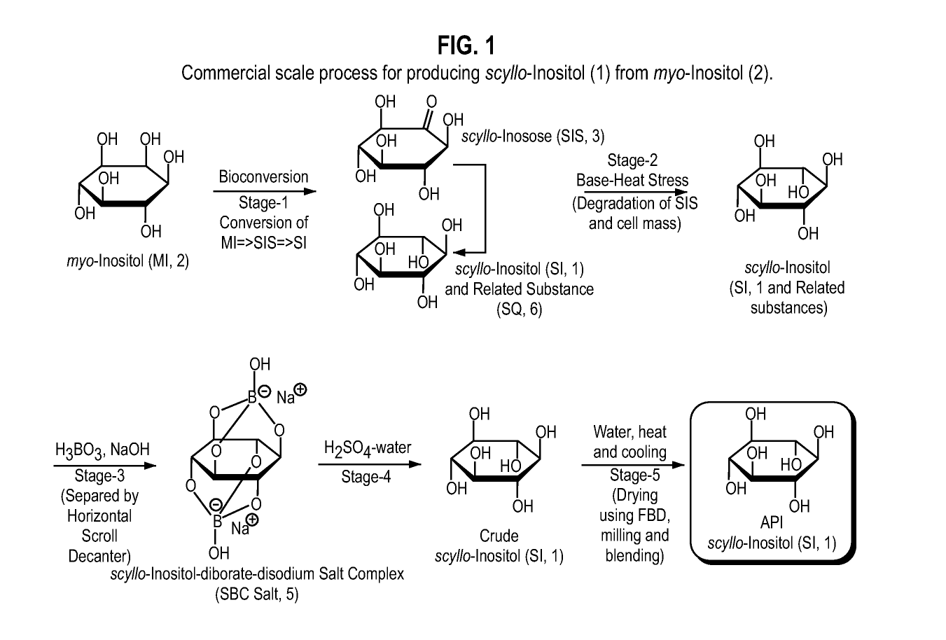

FIG. 2 illustrates one potential method for performing the bioconversion step of the current invention. Specifically, FIG. 2 illustrates a process in which a working stock vial(s) is thawed and inoculated in flask(s) containing medium and incubated with agitation to propagate the culture. The flask(s) or a portion thereof is used to inoculate a Seed Fermentor containing growth medium and incubated for the further propagation of cell mass. One Seed Fermentor or a portion thereof is used to inoculate the Production Fermentor containing production medium including myo-Inositol. Extra Seed fermentors which may be set as a spare are discarded. The fermentation cycle is carried out under aseptic conditions to complete the bioconversion of myo-Inositol (2) to scyllo-Inositol (1) via scyllo-Inosose (3) intermediate. At the end of the fermentation time the myo-Inositol (2) is exhausted, the intermediate scyllo-Inosose (3) is present at g/l quantities and the product, scyllo-Inositol (1) is the major product in the fermentation beer.

FIG. 3 illustrates the typical reaction parameters monitored in the seed fermentors described in FIG. 2, as the culture grows prior to inoculation into the production fermentor. Specifically, FIG. 3 illustrates the parameters such as the Airflow, Backpressure, CER (Carbon-dioxide Evolution Rate), DO (Dissolved Oxygen), OUR (Oxygen Uptake Rate), pH, Agitation, RQ (Respiratory Quotient, CER/OUR), and Temperature.

FIG. 4 illustrates the typical reaction parameters monitored in the production fermentors described in FIG. 2, as the culture bioconverts the myo-Inositol and allowed to react for a designated amount of time. Specifically. FIG. 4 illustrates the parameters such as the Airflow, Backpressure, CER (Carbon-dioxide Evolution Rate), DO (Dissolved Oxygen). OUR (Oxygen Uptake Rate), pH, Agitation, Temperature and Weight.

FIG. 5 illustrates the high-performance liquid chromatography analysis of the conversion of the myo-Inositol (MI) starting product to the scyllo-Inositol (SI) end product. Specifically, FIG. 5 illustrates how scyllo-Inositol is produced over time and how the myo-Inositol and all other undesired by-products such as scyllo-Inosose (SIS) and scyllo-quercitol (SQ) are produced in minimal amounts.

FIG. 6 illustrates a pilot scale process for the production of scyllo-Inositol from myo-Inositol, without the development of the scyllo-Inositol-diborate-disodium salt complex intermediate.

FIG. 7 illustrates a lab scale process for the production of scyllo-Inositol from myo-Inositol, without the development of the scyllo-Inositol-diborate-disodium salt complex intermediate.

### DETAILED DESCRIPTION OF THE INVENTION

The current invention is directed to more efficient and safe processes for the production of scyllo-Inositol from myo-Inositol. The current processes also minimize the production of the undesirable side products that are typically produced by the methods of scyllo-Inositol currently known in the art. In one embodiment, the current invention comprises the following process:

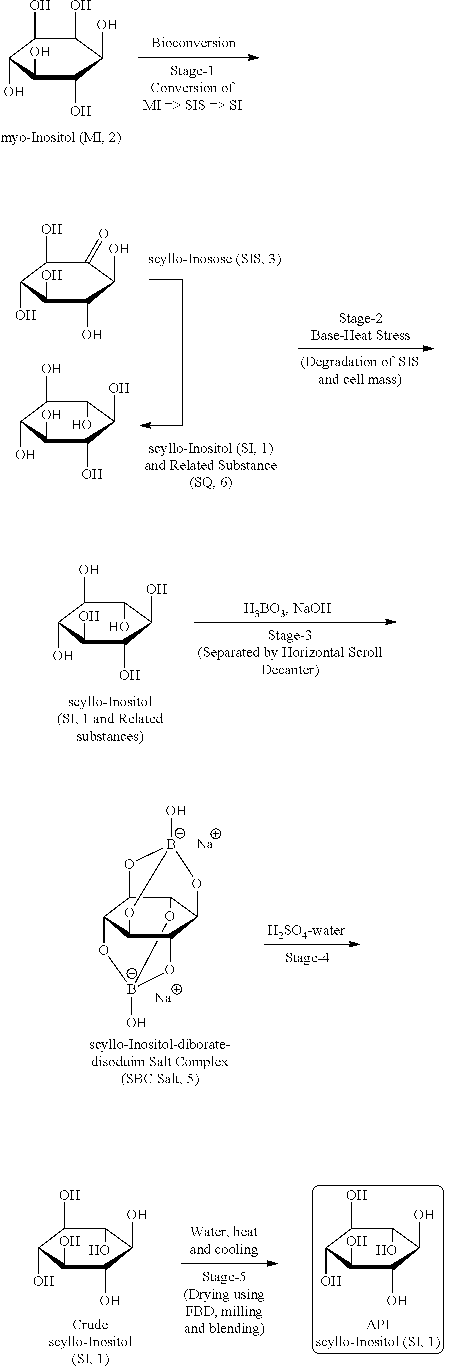

As shown, the current invention is directed to a process for preparing scyllo-Inositol (1) comprising the steps of: (a) subjecting myo-Inositol to a bioconversion process to produce scyllo-Inosose and scyllo-Inositol; (b) reacting the scyllo-Inosose and scyllo-Inositol produced in step (a) with a basic compound and heat to degrade the scyllo-Inosose and lyse the cell mass; (c) converting the scyllo-Inositol of step (b) with boric acid and sodium hydroxide to produce scyllo-Inositol-diborate-disodium salt complex; (d) hydrolyzing the scyllo-Inositol-diborate-disodium salt complex to produce crude scyllo-Inositol; and (e) crystallizing the crude scyllo-Inositol to produce crystalline scyllo-Inositol. The process may be performed as either a batch or continuous process.

The bioconversion process of step (a) can generally be described as the use of live organisms, often microorganisms, to carry out a chemical reaction. In the current invention, microorganisms are used to create a fermentation mixture that is capable of converting myo-Inositol into scyllo-Inositol. It is recognized that the conversion process may produce a mixture of multiple products, including scyllo-Inositol and scyllo-Inosose. Scyllo-Inosose is a structural derivative of scyllo-Inositol that may be converted to the scyllo-Inositol end product when allowed to react for an extended period of time. While the goal of the present invention is to maximize the amount of scyllo-Inositol produced by the current process, it is recognized that some residual scyllo-Inosose may remain after the myo-Inositol is exhausted. The term “residual” scyllo-Inosose is generally defined to include amounts of scyllo-Inosose ranging from about 5% to about 15% by weight of the initial amount of myo-Inositol.

It is recognized that a variety of microorganisms may be used to convert the myo-Inositol into the scyllo-Inosose and scyllo-Inositol, depending on the species desired. Suitable examples of microorganisms that may be incorporated into the bioconversion step include, but are not limited to Acetobacter cerevisiae, Acetobacter malorum, Acetobacter orleanensis, Acetobacter indonesiensis, Acetobacter orientalis, Acetobacter aceti, Acetobacter liquefaciens, Acetobacter pasteurianus, Acetobacter hansenii, Burkholderia andropogonis, Burkholderia caryophylli, and Burkholderia graminis, and combinations thereof. In one embodiment, the microorganism incorporated into the bioconversion process comprises an Acetobacter species. Regardless of the microorganism chosen, the microorganism may include lyophilized species that have previously been freeze-dried, which are typically stored at refrigerated temperatures. Additionally, frozen vials are used which are typically stored at temperatures of less than or equal to −70° C. If a frozen vial is incorporated into the bioconversion process of step (a), the microorganism should be thawed prior to introduction into the flask medium. The bioconversion process of step (a) is typically performed at a temperature ranging from about 20° C. to about 40° C. In another embodiment, step (a) is performed at a temperature ranging from about 26° C. to about 30° C. In a further embodiment, step (a) is performed at a temperature of about 28° C.

In addition to the temperature range of step (a), the bioconversion process is typically initiated at a pH range of approximately 5 to approximately 9. In one embodiment, the initial pH at the beginning of the fermentation process is approximately 7. It is recognized that the pH level may increase or decrease during the process of fermentation, as acidic by-products are produced. It is not uncommon for the pH level to decrease to a level less than pH 4 by the conclusion of the fermentation process. The length of the fermentation process may vary depending on the amount of myo-Inositol converted, as well as the type of organism chosen for the bioconversion process.

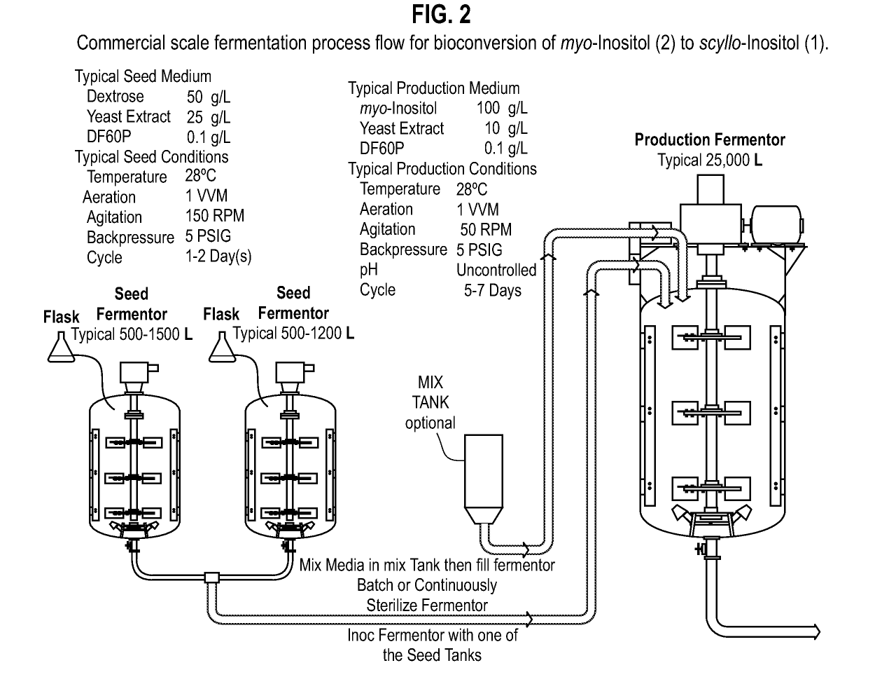

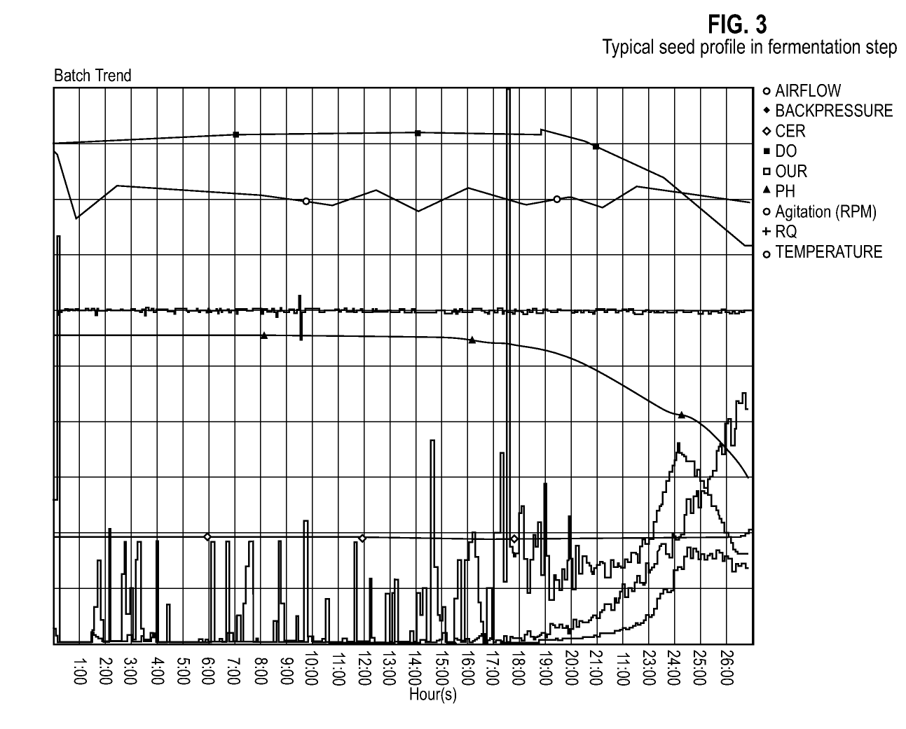

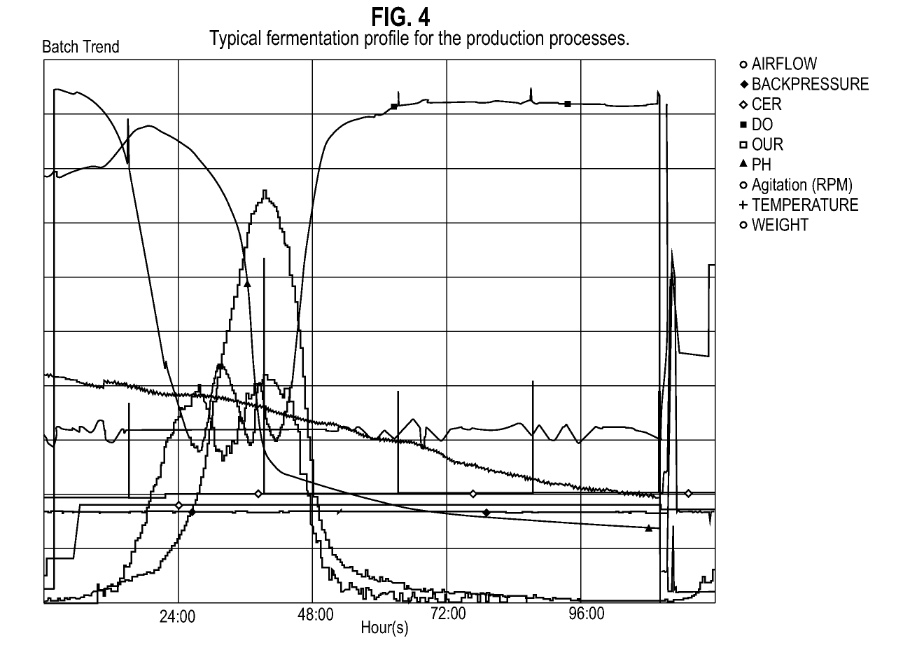

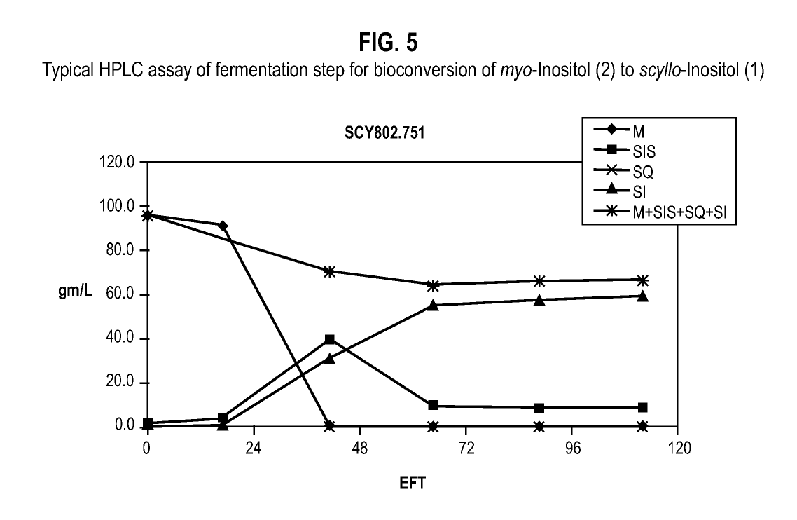

The skilled artisan will appreciate that the bioconversion process may utilize any potential fermentation processes known in the art. In one embodiment, the microorganism may directly inoculate a production fermentation process, whereby the myo-Inositol is converted immediately. In another embodiment, step (a) may comprise several phases of culture expansion including flask and seed fermentor propagation phase, and a production fermentation phase. Under this process, the microorganism is incorporated into one or more seed fermentor tanks, and allowed to grow on the medium for a set amount of time. The growth in the seed fermentors develops a sufficient quantity of the microorganism that is subsequently used to inoculate the production fermentor, where it is allowed to bioconvert the myo-Inositol and begin producing the desired end product. One exemplary embodiment is shown in FIG. 2, which illustrates a system in which a flask(s) and seed fermentor(s) are used to develop sufficient culture mass. Extra flasks and Seed fermentors which may be set as a spare, are discarded. The seed fermentor is then used to inoculate the production fermentor. While the specific conditions incorporated in the seed fermentor may vary depending on the desired product, FIG. 3 provides one exemplary seed profile for the parameters incorporated into the seed fermentor, including the Airflow, Backpressure, CER (Carbon-dioxide Evolution Rate), DO (Dissolved Oxygen), OUR (Oxygen Uptake Rate), pH, Agitation, RQ (Respiratory Quotient, CER/OUR), and Temperature. Additionally, FIG. 4 illustrates one exemplary embodiment, whereby the graph illustrates the various parameters of the production fermentor tank, including the Airflow, Backpressure, CER (Carbon-dioxide Evolution Rate), DO (Dissolved Oxygen), OUR (Oxygen Uptake Rate), pH, Agitation. Temperature and Weight. Moreover, FIG. 5 illustrates the high-performance liquid chromatography (HPLC) analysis for a typical fermentation process similar to step (a). Specifically, as seen in FIG. 5, the myo-Inositol is converted to scyllo-Inositol with only minimal amounts of the other side products formed during fermentation, including scyllo-Inosose and scyllo-quercitol. The skilled artisan will appreciate that the term minimal amounts of side products may be construed to include amounts of the side products less than about 10-15% of the initial amount of myo-Inositol.

Step (b) of the process comprises reacting the scyllo-Inosose and scyllo-Inositol produced in step (a) with a basic compound and heat to degrade the residual scyllo-Inosose. Generally, the basic compound used in step (b) may comprise any compound capable of raising the pH of the fermentation mixture to the desired levels. The basic compound of step (b) may include, but are not limited to sodium hydroxide, sodium carbonate, potassium hydroxide, sodium borohydride, calcium carbonate, and combinations thereof. Step (b) of the process is generally performed at a pH level ranging from about 10 to about 14. In another embodiment step (b) is performed at a pH level ranging from about 12 to about 13. Accordingly, the basic compound incorporated into the reaction of step (b) is typically added in an amount sufficient to raise the pH to the desired level. Thus, the amount of basic compound incorporated into the reaction step (b) can be readily determined by the skilled artisan.

The heating process of step (b) typically comprises any process capable of increasing the temperature of the reaction to facilitate degradation of the scyllo-Inosose and reaction cell mass. The means of heating the reaction mixture may vary depending on the manufacturing limitations of the facilities. In one embodiment, a direct steam injection may be used to increase the temperature of the fermentation mixture. In another embodiment, step (b) may incorporate a heat exchanger to increase the reaction temperature. Regardless of the heating mechanism used, the temperature of the fermentation mixture is generally increased to a level ranging from about 100° C. to about 150° C. In another embodiment, the temperature of the fermentation mixture of step (b) is generally increased to a level ranging from about 115° C. to about 130° C. In a further embodiment, the temperature of the fermentation mixture is increased to a level ranging from about 120° C. to about 125° C.

After the fermentation mixture of step (b) is allowed to react for a sufficient amount of time, the fermentation mixture may be cooled prior to incorporating the reactants of step (c). Generally, step (b) is performed for approximately 5 minutes to approximately 60 minutes. Additionally, in one embodiment, the fermentation mixture of step (b) is cooled to a temperature of less than about 90° C. In another embodiment, the mixture of step (b) is cooled to a temperature of less than about 80° C.

The reaction of step (c) is performed to produce the scyllo-Inositol-diborate-disodium salt complex. Specifically, the scyllo-Inositol produced by step (b) is reacted with boric acid and sodium hydroxide to produce the aforementioned scyllo-Inositol-diborate-disodium salt complex. Generally, the amount of boric acid used in step (c) is sufficient to provide a molar ratio of boric acid to scyllo-Inositol ranging from about 1.5 to about 4. In another embodiment, the molar ratio of boric acid to scyllo-Inositol ranges from about 2 to about 3.5. In a further embodiment, the molar ratio of boric acid to scyllo-Inositol ranges from about 2.5 to about 3. Importantly, step (c) of the current invention does not incorporate sodium chloride in the conversion from scyllo-Inositol to scyllo-Inositol-diborate-disodium salt complex. Sodium chloride is known to be corrosive to stainless steel and other equipment surfaces. As such, the removal of this corrosive reagent improves the efficiency of the process. Step (c) is typically performed at a temperature ranging from about 60° C. to about 80° C. The amount of sodium hydroxide incorporated into the reaction mixture of step (c) is generally sufficient to establish a pH ranging from about 8.5 to about 11. In another embodiment, the amount of sodium hydroxide incorporated into the mixture of step (c) is sufficient to establish a pH ranging from about 9.5 to about 10.5. Thus, the amount of sodium hydroxide incorporated into the reaction step (c) can be readily determined by the skilled artisan. Step (c) may further comprise the subsequent cooling of the mixture to a temperature of less than 30° C.

It is noted that the scyllo-Inositol-diborate-disodium salt complex produced by step (c) is typically separated from the liquid remaining in the reaction mixture, prior to step (d). This process provides a reaction product comprising only the scyllo-Inositol-diborate-disodium salt complex, rather than a mixture of scyllo-Inositol-diborate-disodium salt complex (SBC salt) and solvent. The separation of the scyllo-Inositol-diborate-disodium salt complex is important, as the solvent typically contains many of the impurities that can adversely affect the product yield. Thus, by eliminating the solvent portion, and producing only the solid scyllo-Inositol-diborate-disodium salt complex, the process is able to produce a more pure product, with greater product yield. The separation of the scyllo-Inositol-diborate-disodium salt complex may be performed by any method currently known in the art. In one embodiment, the scyllo-Inositol-diborate-disodium salt complex is passed through a horizontal scroll decanter, such that the scyllo-Inositol-diborate-disodium salt complex is separated without the need for washing or drying of the reaction mixture, providing further cost efficiencies.

Once the scyllo-Inositol-diborate-disodium salt complex is separated, the product of step (c) is hydrolyzed to produce crude scyllo-Inositol. In step (d), the scyllo-Inositol-diborate-disodium salt complex is mixed with water and heated to a temperature ranging from about 30° C. to about 50° C. Generally, the amount of water added in step (d) ranges from about 1 liter of water per kilogram of the SBC salt to about 7 liters of water per kilogram of the SBC salt. In another embodiment, water is added in step (d) in an amount ranging from about 3 to about 5 liters per kilogram of SBC salt. In a further embodiment, water is added in an amount of about 4 liters per kilogram of SBC salt. Additionally, the combination of scyllo-Inositol-diborate-disodium salt complex and water is heated to a temperature ranging from about 36° C. to about 43° C. It is important to note that the current process does not incorporate organic solvents in the hydrolysis process, but instead relies on water as the primary solvent. Organic solvents create issues with regard to potential environmental pollution resulting from disposal of the solvent after use in the process. The use of water as the solvent eliminates the pollutions concerns associated with disposal of an organic solvent.

Once the designated temperature range is achieved, a mineral acid is added to the combination of scyllo-Inositol-diborate-disodium salt complex and water to induce hydrolysis of the scyllo-Inositol-diborate-disodium salt complex. Although the reaction scheme above illustrates the use of sulfuric acid, the skilled artisan will understand that any mineral acid capable of inducing hydrolysis may be used. The mineral acid may include, but is not limited to hydrochloric acid, hydrobromic acid, hydroiodic acid, hypochloric acid, chloric acid, perchloric acid, periodic acid, sulfuric acid, fluorosulfuric acid, nitric acid, phosphoric acid, fluoroantimonic acid, fluoroboric acid, hexafluoroboric acid, and chromic acid. In one embodiment, the mineral acid comprises hydrochloric acid, sulfuric acid, and phosphoric acid. In a further embodiment, the mineral acid comprises sulfuric acid. The amount of mineral acid added to the combination of scyllo-Inositol-diborate-disodium salt complex and water in step (d) is generally an amount sufficient to decrease the pH to a level less than 4. In one embodiment, the amount of mineral acid added to the mixture is an amount sufficient to decrease the pH to a level ranging from about 2 to about 3.5. Thus, the amount of mineral acid incorporated into the reaction step (d) can be readily determined by the skilled artisan.

The reaction product of step (d) may subsequently be cooled to a temperature ranging from about 15° C. to about 26° C. In another embodiment, the reaction product of step (d) may subsequently be cooled to a temperature ranging from about 18° C. to about 24° C. Once the cooling process has completed, the reaction product of step (d) may be subjected to a filtration process to remove excess water from the reaction mixture. The filtration process may include any of those known in the art, and may specifically include centrifugation. It is noted that the reaction product of step (d) is generally not dried after the reaction has concluded. Instead, the crude scyllo-Inositol is processed in step (e) as the wet cake formed from the reaction of step (d). The drying process not only increases the processing time, but may result in the loss of some product.

Step (e) typically comprises the addition of water to the crude scyllo-Inositol produced by step (d), followed by heating of the reaction mixture, and subsequent cooling to produce the crystalline scyllo-Inositol. Generally, water is added to the crude scyllo-Inositol in an amount ranging from about 6 to about 20 liters of water per kilogram of crude scyllo-Inositol. In another embodiment, water is added to the crude scyllo-Inositol in an amount ranging from about 12 to about 18 liters of water per kilogram of crude scyllo-Inositol. In a further embodiment, water is added to the crude scyllo-Inositol in an amount ranging from about 15 to about 17 liters of water per kilogram of crude scyllo-Inositol. Subsequent to the addition of water to the crude scyllo-Inositol produced by step (d), the reaction mixture of water and crude scyllo-Inositol is heated to a temperature ranging from about 70° C. to about 100° C. In another embodiment, the reaction mixture of water and scyllo-Inositol may be heated to a temperature ranging from about 85° C. to about 95° C. The reaction mixture of water and crude scyllo-Inositol produced in step (e) is subsequently cooled to a temperature ranging from about 0° C. to about 25° C. In another embodiment, the reaction mixture of water and crude scyllo-Inositol produced in step (e) is subsequently cooled to a temperature ranging from about 8° C. to about 16° C. Generally, after cooling, the solution of crude scyllo-Inositol and water produced in step (e) is subjected to a solid separation process by either solid filtration or centrifugation, and drying to produce crystalline scyllo-Inositol. Generally, the solid separation process may comprise any process known in the art. In one embodiment, the solid separation process comprises basket centrifugation and scrolled decanter centrifugation. The centrifugation may comprise the use of multiple pre and primary filters to isolate the desired compound. In addition, the drying process may comprise any process for drying currently known in the art. In one embodiment, the drying method comprises the use of hot air in a fluid bed dryer, a tray dryer, a tumble dryer, and a unidryer.

The process for producing scyllo-Inositol, as described in this embodiment, provides multiple benefits compared to methods known within the art. The methods of this embodiment do not require the use of organic solvents, which are difficult to dispose of, and may have an adverse effect on the environment. Moreover, the processes of the current embodiment also do not require the use of certain corrosive reactants such as sodium chloride. In addition to these benefits, the process results in an unexpectedly high yield of scyllo-Inositol. Generally, the process results in scyllo-Inositol yields ranging from approximately 20% to approximately 50% based on the initial amount of myo-Inositol used in the process. In another embodiment, the scyllo-Inositol yield ranges from approximately 25% to approximately 35% based on the initial amount of myo-Inositol used in the process.

In an alternative embodiment, the current invention encompasses a process in which the scyllo-Inositol-diborate-disodium salt complex is not formed, such that the crude scyllo-Inositol created by the bioconversion step, and the subsequent degradation of scyllo-Inosose by exposure to a basic compound and heat, is followed by crystallization of the compound. This embodiment is illustrated by the following steps:

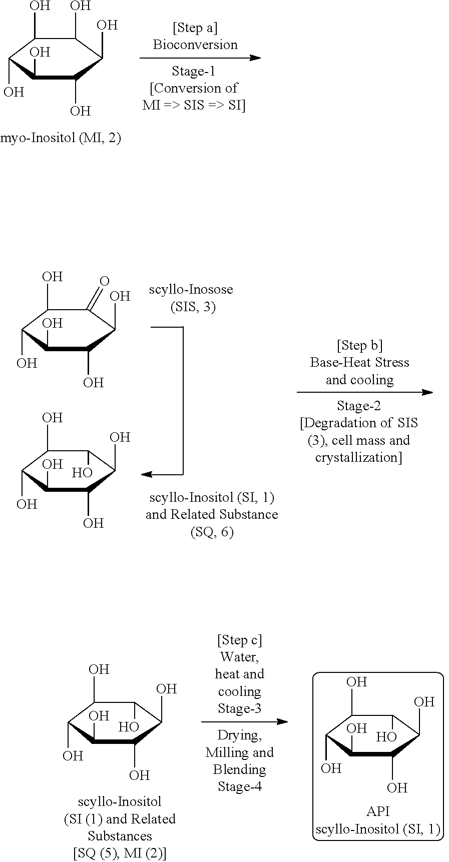

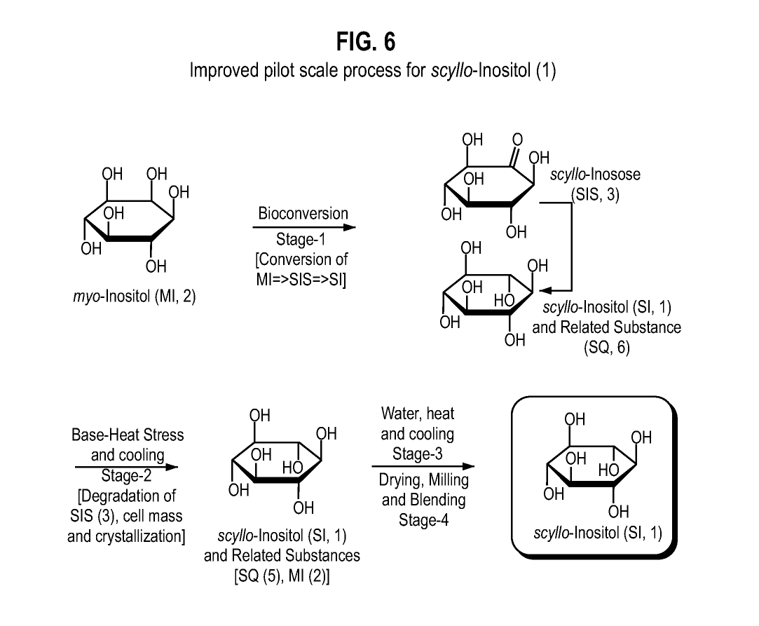

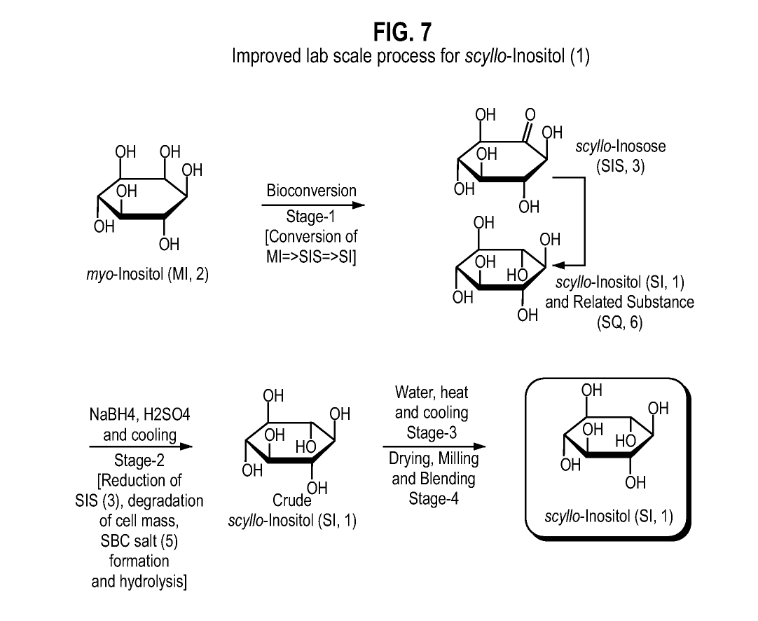

As illustrated, this embodiment comprises a process for preparing scyllo-Inositol (1) comprising the steps of: (a) subjecting myo-Inositol to a bioconversion process to produce scyllo-Inosose and scyllo-Inositol; (b) reacting the scyllo-Inosose and scyllo-Inositol produced in step (a) with a basic compound and heat to degrade the scyllo-Inosose; and (c) crystallizing the crude scyllo-Inositol to produce crystalline scyllo-Inositol. This embodiment of the current invention is illustrated in FIGS. 6 and 7. It is noted that steps (a) and (c) of the current embodiment are similar to steps (a) and (e), respectively, of the embodiment previously described. As such, the parameters and considerations pertaining to steps (a) and (e) are hereby referenced and incorporated for steps (a) and (c), respectively, of the current embodiment.

Step (b) of the current embodiment is directed to a process for degrading scyllo-Inosose. Similar to the previous embodiment, the basic compound used to degrade the scyllo-Inosose is generally one that is capable of increasing the pH of the reaction mixture. Suitable examples of the basic compounds that may be incorporated include, but are not limited to sodium hydroxide, sodium carbonate, potassium hydroxide, sodium borohydride, calcium carbonate, and combinations thereof. In one embodiment, the basic compound comprises sodium hydroxide. In a further embodiment, the basic compound comprises sodium borohydride.

The temperature and pH range of the reaction of step (b) is generally dependent upon the basic compound utilized to degrade the scyllo-Inosose. In one embodiment of the process for producing scyllo-Inositol without the formation of scyllo-Inositol-diborate-disodium salt complex, sodium hydroxide is utilized as the basic compound of step (b), and the pH of the reaction mixture is increased to a level ranging from about 12 to about 13. In this embodiment, the temperature of the reaction mixture is increased to a level ranging from about 100° C. to about 150° C., and specifically to a temperature ranging from about 115° C. to about 130° C.

In another embodiment of the process for producing scyllo-Inositol without the formation of scyllo-Inositol-diborate-disodium salt complex, sodium borohydride may be selected as the basic compound used in step (b). In this embodiment, the reaction mixture is typically adjusted to a pH level ranging from about 6 to about 8. The sodium borohydride may be added to the reaction mixture at a temperature ranging from about 50° C. to about 70° C. In another embodiment, the sodium borohydride may be added to the reaction mixture at a temperature of about 60° C. Subsequently, the resulting mixture of scyllo-Inositol, scyllo-Inosose, and sodium borohydride is acidified using sulfuric acid to a pH level of approximately 3.5 or less. The acidified reaction mixture may then be heated to a temperature ranging from about 80° C. to about 100° C. In one embodiment, the acidified reaction mixture may be heated to a temperature of about 90° C. This specific embodiment is illustrated in FIG. 7.

Regardless of the basic compound used in step (b), after the reaction mixture of step (b) is heated, it is subsequently cooled in preparation for the crystallization process of step (c). The reaction mixture of step (b) may be cooled to a temperature ranging from about 0° C. to about 25° C. In another embodiment, the reaction mixture of step (b) is subsequently cooled to a temperature ranging from about 8° C. to about 16° C.

The current embodiment incorporating fewer process steps than the previous embodiment provides a process for producing scyllo-Inositol without the use of organic acids or certain corrosive reactants. These changes to the processes known in the prior art provide a more efficient and environmentally conscious method of manufacturing scyllo-Inositol.

The compounds and processes of the invention will be better understood by reference to the following examples, which are intended as an illustration of and not a limitation upon the scope of the invention. Each example illustrates at least one method of preparing various intermediate compounds and further illustrates each intermediate utilized in the overall process. These are certain preferred embodiments, which are not intended to limit the present invention's scope. On the contrary, the present invention covers all alternatives, modifications, and equivalents as can be included within the scope of the claims, routine experimentation, including appropriate manipulation of the reaction conditions, reagents used, and sequence of the bioconversion and synthetic route, protection of any chemical functionality that can be compatible with the reaction conditions, and deprotection at suitable points in the reaction sequence of the method are included within the scope of the present invention.

### EXAMPLES

### Example 1

### Conversion of Myo-Inositol (2) to Scyllo-Inositol (1) and Degradation of Scyllo-Inosose (3) and Cell Mass

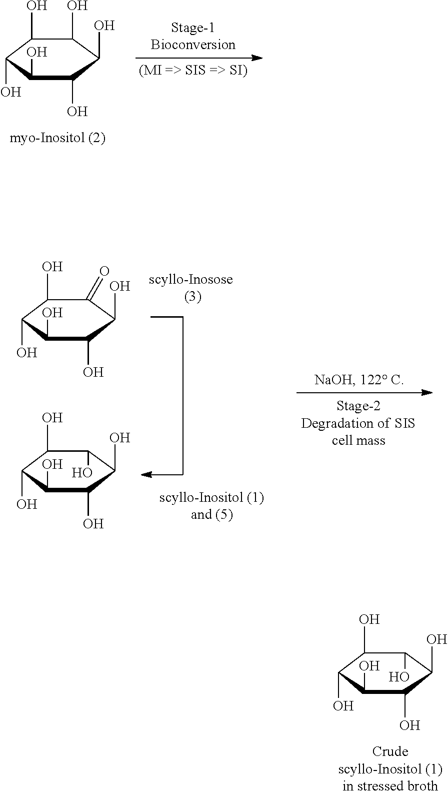

Cell Banks and Working Stocks were made from lyo Acetobactor Species in 20 mL vials containing culture and cryoprotective agent(s), and they are stored at −70° C. or colder temperature. A working stock is thawed an inoculated in 1.5 liters flask medium in a 4 L Flask. It is then incubated at 28±2° C. temperature for approximately 24 h at 240±10 rpm and the Optical Density (OD) and residual glucose were measured. The flask or a portion thereof is used to inoculate a Seed Fermentor at 0.01 to 0.1% for the propagation of cell mass. The Seed Fermentor is controlled at 28° C., agitation of approximately 150 rpm and aeration of approximately 1 VVM for a cycle of 24-30 h. The Seed Fermentor or a portion thereof is used to inoculate the Production Fermentor at 1-5% at 2500 Kg scale of myo-Inositol (2). The fermentation conditions are as follows. Temperature: 28° C., Agitation: 50 rpm, Aeration: 0.5 VVMO-5 h and ramped to 1 VVM, and Backpressure: 5 psig. The pH is not controlled but monitored to drop from a starting pH of approximately 7 at the beginning to below 4 at the end of the fermentation. The fermentation cycle was carried out under aseptic conditions for 5 days to complete the bioconversion of myo-Inositol (2) to scyllo-Inositol (1) via scyllo-Inosose (3) intermediate. At the end of 5 day fermentation time the myo-Inositol (2) is exhausted, the scyllo-Inosose (3) is present at approximately 10-15 g/L and the product, scyllo-Inositol (1) is measured to be approximately 55-60 g/L. The pH of the resulting fermentation broth, containing cell mass, scyllo-Inositol (1) and small amount of scyllo-Inosose (3) was adjusted to about 12-13 using 25% aqueous sodium hydroxide solution and the broth was heated to 120-125° C. for NLT 10 minutes using steam. The resulting dark brown stressed broth was cooled to below 80° C. temperature and a sample of the stressed broth was tested to determine the amount of scyllo-Inositol (1) present as g/L. Based on assay, the total amount of scyllo-Inositol was estimated to be 1377 Kg present in the stressed broth.

### Example 2

### Selective Conversion of Scyllo-Inositol (1) in Stressed Broth to Scyllo-Inositol Diborate-Disodium Salt Complex (SBC Salt, 5) and Separation Using Horizontal Scroll Decanter

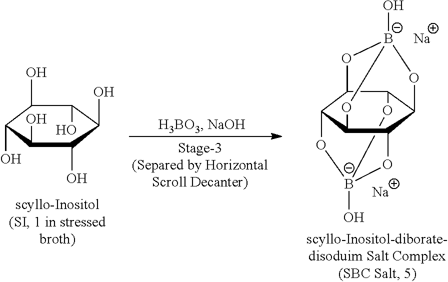

In a separate SS-reactor, 1323 Kg of boric acid (2.8 equiv.) was suspended in 2065 L of water [1.5 L/1 Kg of scyllo-Inositol (1)] and heated to NLT 60° C. temperature. The resulting slurry was transferred into the fermentation vessel, containing base-heat stressed broth and scyllo-Inositol (1). An additional amount of water [1377 L, 1.0 L/1 Kg of scyllo-Inositol (1)] was charged to the boric acid reactor, heated to NLT 60° C. temperature and the solution was transferred to fermentation vessel, containing stressed broth. The total volume of contents in fermentation vessel, containing stressed broth, scyllo-Inositol (1) and boric acid was measured to be 32500 L, which was further adjusted to 34414 L by addition of 1914 L of water, to maintain Stage-3 starting volume of 4 L/Kg of scyllo-Inositol (1) in stressed booth. Temperature of the mixture was adjusted to 60-80° C. and 25% aqueous sodium hydroxide solution was charged to adjust the pH of the mixture to 9.5-10.5 over NLT 1 h with agitation. The resulting slurry containing to scyllo-Inositol-diborate-disodium Salt Complex (SBC Salt, 5) was mixed for NLT 3 h while maintaining the temperature of the reaction mixture between 60-80° C. and then cooled to below 30° C. temperature.

In a separate SS-reactor, 2753 L of water [2.0 L/1 Kg of scyllo-Inositol (1)] was taken and mixed gently at 15-25° C. temperature. The scyllo-Inositol-diborate-disodium Salt Complex (SBC Salt, 5) slurry from the fermentation reactor was passed through an Horizontal Scroll Decanter (CA-225) at about 2200 RPM and a flow rate of 20-100 L/h to separate the scyllo-Inositol-diborate-disodium Salt Complex (SBC Salt, 5) from liquid, while dropping the solids directly into the mixing water (2753 L) in a separate SS-reactor. The dark brown liquid waste from Horizontal Scroll Decanter (CA-225) was periodically checked to make sure that no scyllo-Inositol-diborate-disodium Salt Complex (SBC Salt, 5) solids were present. The Horizontal Scroll Decanter RPM and slurry flow rate were adjusted, as needed, to ensure that no solids were present until all the slurry form fermentation reactor was passed through and all scyllo-Inositol-diborate-disodium Salt Complex (SBC Salt, 5) was separated and dropped in to the water in SS-reactor.

### Example 3

### Hydrolysis of Scyllo-Inositol-Diborate-Disodium Salt Complex (SBC Salt, 5) to Scyllo-Inositol (1) and Isolation of Crude Scyllo-Inositol (1)

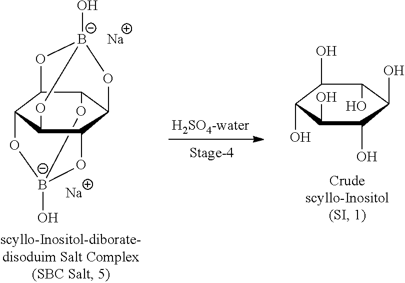

An additional amount of water [2753 L, [2.0 L/1 Kg of scyllo-Inositol (1)] was charged to the SS-reactor, containing scyllo-Inositol-diborate-disodium Salt Complex (SBC Salt, 5) in water and heated the mixture to 36-43° C. temperature. To this suspension, concentrated sulfuric acid was charged slowly over NLT 1 h and the pH of the SBC salt suspension was adjusted 2.0-3.5, while maintaining the temperature between 36-43° C. with vigorous agitation. After the addition of sulfuric acid was complete and a stable pH of 2.0-3.5 was achieved, the resulting scyllo-Inositol (1) slurry was mixed for NLT 4 h while maintaining the temperature between 36-43° C. The mixture was cooled to 18-24° C. temperature and the crude scyllo-Inositol (1) was isolated as a wet cake (1746 Kg) by filtration via basket centrifugation and collected in the crude product in drums. Multiple composite samples of crude scyllo-Inositol (1) product, each from about 3-4 drums was tested for Loss on Drying (LOD) and the scyllo-Inositol (1) on a dry basis was calculated to 1370 Kg, before proceeding next stage.

### Example 4

### Crystallization of Crude Scyllo-Inositol (1) Wet Cake and Isolation of Scyllo-Inositol (1)

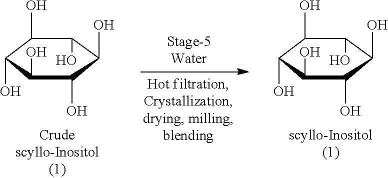

The crude scyllo-Inositol (1) wet cake was crystallized, dried, milled in portions, and the scyllo-Inositol (1) product was staged in a blender till all sub batches of crude scyllo-Inositol (1) processing was complete. Thus, a maximum of 220 Kg based on dry weight of crude scyllo-Inositol (1) wet cake was charged to SS-reactor containing 3600 L of purified water [16.5 L/1 kg of crude scyllo-Inositol (1)] and the suspension was heated to 85-95° C. for NLT 15 minutes to dissolve all solids. The resulting clear and hot scyllo-Inositol (1)-water solution was filtered through sets of pre and primary filters [cotton (1 μm rated) depth pre-filter followed by polyethersulfone (PES) filter with two pore size membranes (1.0 μm, absolute and 0.22 μm, absolute)] into separate SS-crystallizer. After the filtration was complete, the clear brown solution in the SS-crystallizer was heated to 85-95° C. for NLT 10 minutes and gradually cooled to 8-16° C. over NLT 3 hours. The resulting slurry was filtered via centrifugation and the color less scyllo-Inositol (1) wet cake was washed with purified chilled water at NMT 100 L per centrifuge load. The wet scyllo-Inositol (1) was dried using hot air in a Fluidized Bed Dryer (FBD) for NLT 1 h with an inlet air temperature 90° C. until a composite sample of scyllo-Inositol (1) meets Loss on Drying (LOD) test with a limit of NLT 1.0 w/w %. The dried scyllo-Inositol (1) product is milled using Comil containing ˜840 μm sieve and all sub-batches are combined in the Beardsley & Piper blender. The combined scyllo-Inositol (1) product is blended at 30 RPM for NLT 15 minutes and a sample of scyllo-Inositol (1) product tested for Loss on Drying (LOD) test with limit of NLT 1.5 w/w %, which was then packaged in poly-lined drums to yield 722.6 Kg of scyllo-Inositol (1) in 28.9% overall yield. The scyllo-Inositol was filtered via a basket centrifuge.

### Example 5

### Conversion of Myo-Inositol (2) to Scyllo-Inositol (1), Degradation of SIS and Cell Mass and Direct Isolation of Crude Scyllo-Inositol (1)

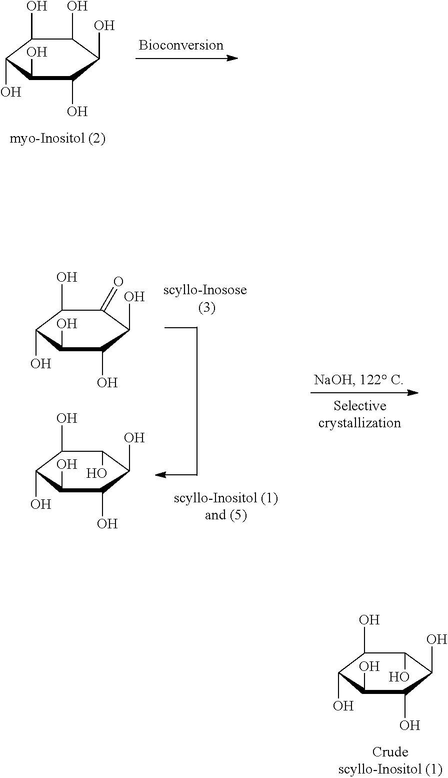

A working stock is thawed and inoculated in 1.5 liters flask medium in a 4 L Flask. It is then incubated at 28±2° C. temperature for approximately 24 h at 240±10 rpm and the Optical Density (OD) and residual glucose were measured. The flask or a portion thereof is used to inoculate a Seed Fermentor at 0.01 to 0.1% for the propagation of cell mass. The Seed Fermentor is controlled at 28° C., agitation of approximately 150 rpm and aeration of approximately 1 VVM for a cycle of 24-30 h. The Seed Fermentor or a portion thereof is used to inoculate the Production Fermentor at 1-5% at the 40 Kg scale of myo-Inositol (2). The fermentation conditions are as follows. Temperature: 28° C., Agitation: 100 rpm, Aeration: 0.5 VVM 0-5 h and ramped to 1 VVM, and Backpressure: 5 psig. The pH is not controlled but monitored to drop from a starting pH of approximately 7 at the beginning to below 4 at the end of the fermentation. The fermentation cycle was carried out under aseptic conditions for 5 days to complete the bioconversion of myo-Inositol (2) to scyllo-Inositol (1) via scyllo-Inosose (3) intermediate. At the end of 5 day fermentation time the myo-Inositol (2) is exhausted, the scyllo-Inosose (3) is present at approximately 10-15 g/L and the product, scyllo-Inositol (1) is measured to be approximately 55-60 g/L. The pH of the fermentation broth was adjusted to about 12-13 using sodium hydroxide solution and the mixture was heated to 120-125° C. for NLT 10 minutes. The resulting stress broth was cooled to below 15° C. over NLT 4 hours. The resulting slurry was filtered via basket centrifugation and the wet cake was washed with chilled water (8 kg) to afford 17.6 kg of scyllo-Inositol (1) as a pale brown crystalline wet solid.

### Example 6

### Crystallization of Crude Scyllo-Inositol (1) Wet Cake and Isolation of Scyllo-Inositol (1)

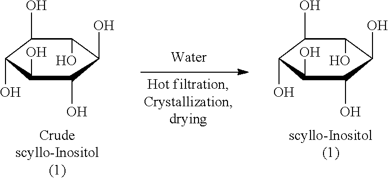

Crude scyllo-Inositol (1) wet cake (12.9 kg and 11.0 kg based on dry weight) was charged to a reactor containing water (179 kg) and the suspension was heated to NLT 90° C. for NLT 15 minutes. The resulting clear solution was filtered through 0.2 μm membrane filter under pressure. The filtered solution was cooled to below 15° C. over NLT 3 hours and the resulting slurry was filtered via centrifugation and the wet cake was washed with chilled water (1.5 kg) to afford 17.6 kg of scyllo-Inositol (1) as a colorless (white) crystalline wet solid. The wet scyllo-Inositol (1) was dried in a vacuum oven at 100-104° C. for NLT 12 hours to produce 4.75 kg of dry scyllo-Inositol (1).

### Example 7

### Conversion of Myo-Inositol (2) to Scyllo-Inositol (1) and Isolation of Crude Scyllo-Inositol (1)

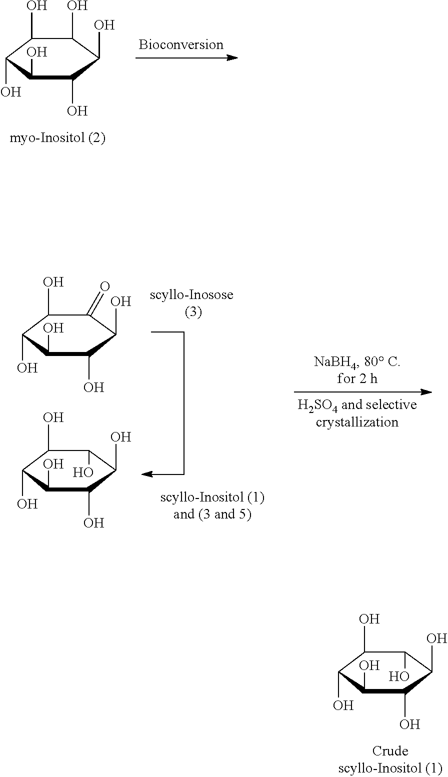

A portion of the fermentation broth (2.2 L), which was prepared as described in Example 1, was heated to 60° C. and the pH was adjusted to about 7 using sodium hydroxide solution. Sodium borohydride (14.98 g) was added in portion wise and the mixture was held at for 60° C. for NLT 3 hours. The resulting mixture was acidified to a pH NMT of 3.5 using sulfuric acid and heated to 90° C. for NLT 15 minutes to form a clear solution and then cooled to below 15° C. over NLT 4 hours. The resulting slurry was filtered and the wet cake was washed with chilled water (0.1 kg) to afford 0.082 kg of scyllo-Inositol (1) as a pale brown crystalline wet solid.

### Example 8

### Crystallization of Crude Scyllo-Inositol (1) Wet Cake and Isolation of Scyllo-Inositol (1)

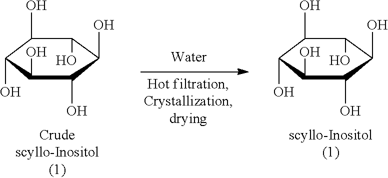

Crude scyllo-Inositol (1) wet cake (0.082 kg and 0.081 kg based on dry weight) was charged to a glass reactor containing water (1.35 kg) and the suspension was heated to NLT 90° C. for NLT 15 minutes. The resulting clear solution was filtered through 0.2 μm membrane filter using a pump. The filtered solution was cooled to below 15° C. over NLT 3 hours and the resulting slurry was filtered and the wet cake was washed with chilled water (0.05 Kg) to afford wet scyllo-Inositol (1), which was dried in a vacuum oven at 100-104° C. for NLT 12 hours to afford 0.047 kg of scyllo-Inositol (1) as a white crystalline solid.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof.

What is claimed is:

1. A process for preparing scyllo-Inositol (1) comprising the steps of:

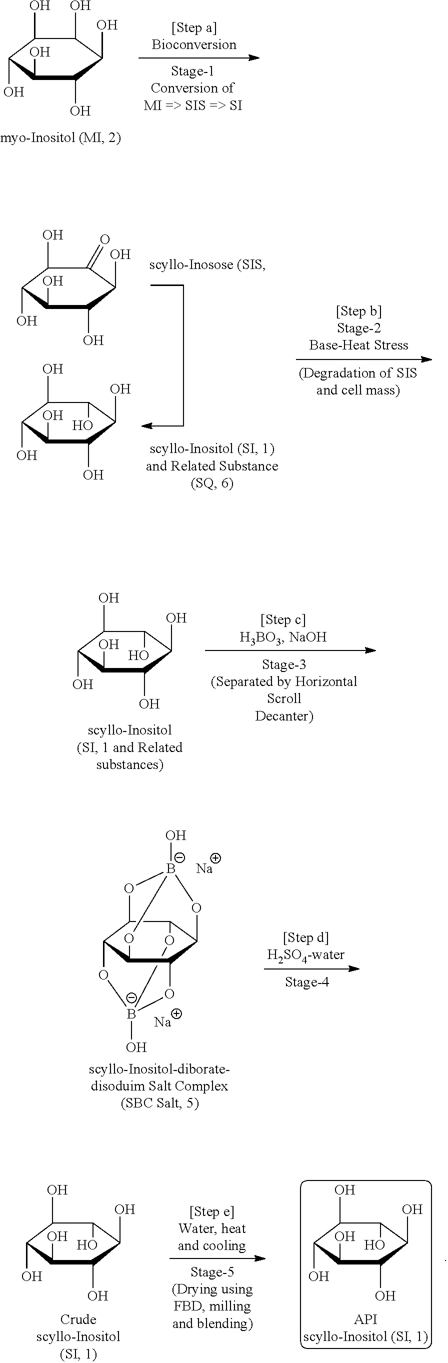

2. A process for preparing scyllo-Inositol (1) comprising the steps of:
a. subjecting myo-Inositol to a bioconversion process to produce scyllo-Inosose and scyllo-Inositol;
b. reacting the scyllo-Inosose and scyllo-Inositol produced in step (a) with a basic compound and heat to degrade the scyllo-Inosose and lyse the cell mass;
c. converting the scyllo-Inositol of step (b) with boric acid and sodium hydroxide to produce scyllo-Inositol-diborate-disodium salt complex;
d. hydrolyzing the scyllo-Inositol-diborate-disodium salt complex with sulfuric acid and water to produce crude scyllo-Inositol; and
e. crystallizing the crude scyllo-Inositol to produce crystalline scyllo-Inositol.

3. The process of claim 2, wherein the bioconversion step comprises creating a fermentation broth, whereby the fermentation is facilitated by a microorganism capable of converting the myo-Inositol into scyllo-Inositol.

4. The process of claim 2, wherein the microorganism capable of converting myo-Inositol into scyllo-Inositol comprises Acetobacter cerevisiae, Acetobacter malorum, Acetobacter orleanensis, Acetobacter indonesiensis, Acetobacter orientalis, Acetobacter aceti, Acetobacter liquefaciens, Acetobacter pasteurianus, Acetobacter hansenii, Burkholderia andropogonis, Burkholderia caryophylli, and Burkholderia graminis.

5. The process of claim 3, wherein the microorganism capable of converting the myo-Inositol into scyllo-Inositol comprises a lyophilized and/or a frozen culture.

6. The process of claim 3, wherein step (a) is performed at a temperature ranging from about 20° C. to about 40° C.

7. The process of claim 3, wherein step (a) is performed at a temperature ranging from about 26° C. to about 30° C.

8. The process of claim 2, wherein the basic compound of step (b) comprises sodium hydroxide, potassium hydroxide, sodium carbonate, calcium carbonate, and combinations thereof.

9. The process of claim 8, wherein the amount of basic compound added to the fermentation broth in step (b) is an amount sufficient to increase the pH of the fermentation broth to a level ranging from about 10 to about 13.

10. The process of claim 8, wherein the amount of basic compound added to the fermentation broth in step (b) is an amount sufficient to increase the pH of the fermentation broth to a level ranging from about 12 to about 13.

11. The process of claim 2, wherein step (b) comprises a direct steam injection to increase the temperature of the fermentation broth.

12. The process of claim 11, wherein the temperature of the fermentation broth is increased to a level ranging from about 100° C. to about 150° C.

13. The process of claim 11, wherein the temperature of the fermentation broth is increased to a level ranging from about 115° C. to about 130° C.

14. The process of claim 12, wherein after the fermentation broth is heated to a temperature ranging from about 100° C. to about 150° C., the broth is cooled to a temperature less than about 80° C.

15. The process of claim 2, wherein the reaction of step (c) is performed at a temperature ranging from about 60° C. to about 80° C.

16. The process of claim 2, wherein the amount of sodium hydroxide incorporated into the broth of step (c) is sufficient to establish a pH ranging from about 8.5 to about 11.

17. The process of claim 2, wherein the amount of sodium hydroxide incorporated into the broth of step (c) is sufficient to establish a pH ranging from about 9.5 to about 10.5.

18. The process of claim 15, wherein step (c) further comprises the subsequent cooling of the broth to a temperature of less than 30° C.

19. The process of claim 2, wherein the scyllo-Inositol-diborate-disodium salt complex produced by step (c) is passed through a horizontal scroll decanter prior to step (d).

20. The process of claim 2, wherein the combination of scyllo-Inositol-diborate-disodium salt complex and water in step (d) is heated to a temperature ranging from about 30° C. to about 50° C., prior to addition of sulfuric acid.

21. The process of claim 2, wherein the combination of scyllo-Inositol-diborate-disodium salt complex and water in step (d) is heated to a temperature ranging from about 36° C. to about 43° C., prior to addition of sulfuric acid.

22. The process of claim 20, wherein the amount of sulfuric acid added to the combination of scyllo-Inositol-diborate-disodium salt complex and water in step (d) is sufficient to decrease the pH to a level ranging from about 2 to about 3.5.

23. The process of claim 22, wherein the reaction product of step (d) is subsequently cooled to a temperature ranging from about 15° C. to about 26° C.

24. The process of claim 22, wherein the reaction product of step (d) is subsequently cooled to a temperature ranging from about 18° C. to about 24° C.

25. The process of claim 2, wherein step (e) comprises the addition of water to the crude scyllo-Inositol, followed by heating of the reaction mixture, and subsequent cooling to produce the crystalline scyllo-Inositol.

26. The process of claim 2, wherein subsequent to the addition of water to the crude scyllo-Inositol produced by step (d), the reaction mixture of water and crude scyllo-Inositol is heated to a temperature ranging from about 70° C. to about 100° C.

27. The process of claim 2, wherein subsequent to the addition of water to the crude scyllo-Inositol produced in step (d), the reaction mixture of water and scyllo-Inositol is heated to a temperature ranging from about 85° C. to about 95° C.

28. The process of claim 25, wherein the reaction mixture of water and crude scyllo-Inositol produced in step (e) is subsequently cooled to a temperature ranging from about 8° C. to about 16° C.

29. The process of claim 27, wherein the cooled solution of crude scyllo-Inositol and water produced in step (e) is subjected to a solid separation process and drying to produce crystalline scyllo-Inositol.

30. The process of claim 28, wherein the solid separation process comprises basket centrifugation and scrolled decanter centrifugation.

31. The process of claim 29, wherein the drying process comprises the use of hot air in a fluid bed dryer, a tray dryer, a tumble dryer, and a unidryer.

32. A process for preparing scyllo-Inositol (1) comprising the steps of:

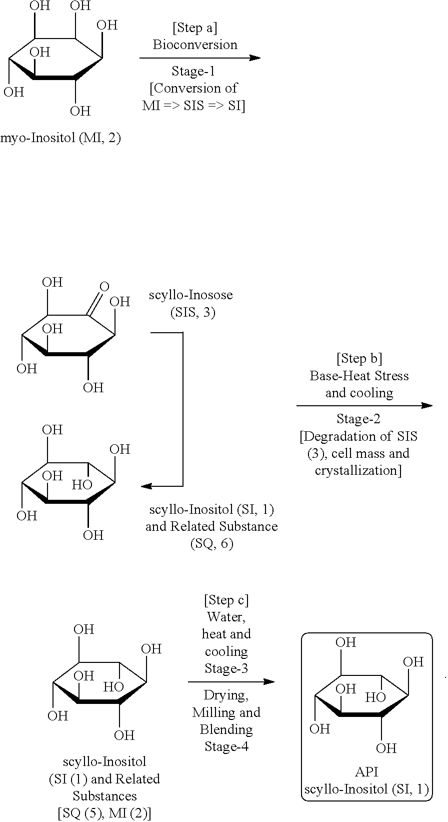

33. A process for preparing scyllo-Inositol (1) comprising the steps of:
a. subjecting myo-Inositol to a bioconversion process to produce scyllo-Inosose and scyllo-Inositol;
b. reacting the scyllo-Inosose and scyllo-Inositol produced in step (a) with a basic compound and heat to degrade the scyllo-Inosose; and lyse the cell mass
c. crystallizing the crude scyllo-Inositol to produce crystalline scyllo-Inositol.

34. The process of claim 33, wherein the bioconversion step comprises creating a fermentation mixture, whereby the fermentation is facilitated by a microorganism capable of converting the myo-Inositol into scyllo-Inositol.

35. The process of claim 33, wherein the microorganism capable of converting myo-Inositol into scyllo-Inositol comprises Acetobacter cerevisiae, Acetobacter malorum, Acetobacter orleanensis, Acetobacter indonesiensis, Acetobacter orientalis, Acetobacter aceti, Acetobacter liquefaciens, Acetobacter pasteurianus, Acetobacter hansenii, Burkholderia andropogonis, Burkholderia caryophylli, and Burkholderia graminis.

36. The process of claim 34, wherein the microorganism capable of converting the myo-Inositol into scyllo-Inositol comprises a lyophilized culture and a frozen culture.

37. The process of claim 34, wherein step (a) is performed at a temperature ranging from about 20° C. to about 40° C.

38. The process of claim 34, wherein step (a) is performed at a temperature ranging from about 26° C. to about 30° C.

39. The process of claim 33, wherein the basic compound of step (b) comprises sodium hydroxide, potassium hydroxide, sodium carbonate, sodium borohydride, calcium carbonate, and combinations thereof.

40. The process of claim 39, wherein the basic compound of step (b) comprises sodium hydroxide.

41. The process of claim 40, wherein the amount of basic compound added to the fermentation mixture in step (b) is an amount sufficient to increase the pH of the fermentation mixture to a level ranging from about 10 to about 13.

42. The process of claim 40, wherein the amount of basic compound added to the fermentation mixture in step (b) is an amount sufficient to increase the pH of the fermentation mixture to a level ranging from about 12 to about 13.

43. The process of claim 39, wherein the basic compound of step (b) comprises sodium borohydride.

44. The process of claim 40, wherein the amount of basic compound added to the fermentation mixture in step (b) is an amount sufficient to increase the pH of the fermentation mixture to a level ranging from about 6 to about 8.

45. The process of claim 33, wherein step (b) comprises a direct steam injection to increase the temperature of the fermentation mixture.

46. The process of claim 45, wherein the temperature of the fermentation mixture is increased to a level ranging from about 100° C. to about 150° C.

47. The process of claim 45, wherein the temperature of the fermentation mixture is increased to a level ranging from about 115° C. to about 130° C.

48. The process of claim 45, wherein the temperature of the fermentation mixture is increased to a level ranging from about 50° C. to about 70° C.

49. The process of claim 45, wherein the temperature of the fermentation mixture is increased to a level ranging from about 85° C. to about 95° C.

50. The process of claim 33, wherein the reaction product of step (b) is subsequently cooled to a temperature ranging from about 15° C. to about 26° C.

51. The process of claim 33, wherein the reaction product of step (b) is subsequently cooled to a temperature ranging from about 18° C. to about 24° C.

52. The process of claim 33, wherein step (b) further comprises subsequently acidifying the fermentation mixture by the addition of sulfuric acid.

53. The process of claim 52, wherein the amount of sulfuric acid added to the fermentation mixture is an amount sufficient to decrease the pH of the fermentation mixture to a level of about 3.5 or less.

54. The process of claim 53, wherein after the pH of the fermentation mixture is decreased to a level of about 3.5 or less, the fermentation mixture is subsequently heated to a temperature ranging from about 80° C. to about 100° C.

55. The process of claim 33, wherein step (c) comprises the addition of water to the crude scyllo-Inositol, followed by heating of the reaction mixture, and subsequent cooling to produce the crystalline scyllo-Inositol.

56. The process of claim 33, wherein subsequent to the addition of water to the crude scyllo-Inositol produced by step (b), the reaction mixture of water and crude scyllo-Inositol is heated to a temperature of greater than 80° C.

57. The process of claim 56, wherein the reaction mixture of water and crude scyllo-Inositol produced in step (c) is subsequently cooled to a temperature ranging from about 8° C. to about 16° C.

58. The process of claim 57, wherein the cooled solution of crude scyllo-Inositol and water produced in step (e) is subjected to a solid separation process and drying to produce crystalline scyllo-Inositol.

59. The process of claim 58, wherein the solid separation process comprises basket centrifugation and scrolled decanter centrifugation.

60. The process of claim 58, wherein the drying process comprises the use of hot air in a fluid bed dryer, a tray dryer, a tumble dryer, and a unidryer.
